(19) United States
(12) Patent Application Publication
(10) Pub. No.: US 2023/0399275 A1
(43) Pub. Date: Dec. 14, 2023

(54) Method and Plant for Producing Olefins

(71) Applicant: LINDE GMBH, Pullach (DE)

(72) Inventors: Torben Hofel, München (DE); Helmut Fritz, München (DE)

(21) Appl. No.: 18/250,625

(22) PCT Filed: Oct. 29, 2021
(86) PCT No.: PCT/EP2021/080124

§ 371 Date: Apr. 26, 2023

(30) Foreign Application Priority Data
Oct. 29, 2020 (EP) 20204783.3

(51) Int. Cl. C07C 4/10; C07C 4/04

(52) U.S. Cl. CPC C07C 4/10; C07C 4/04

(57) ABSTRACT

A process for producing olefins includes subjecting a first feed stream to dehydrogenation to obtain a first product stream, and subjecting part of the first product stream to a first separation sequence. The first separation sequence comprises a first separation step to which part of the first product stream is fed and in which a gas fraction and a liquid fraction are formed. The first separation sequence comprises a second separation step to which part of the liquid fraction is fed and in which the transfer fraction is formed. A second feed stream is subjected to steam cracking to obtain a second product stream. part of which is subjected to crude gas compression and thereafter to a second separation sequence. In the first separation sequence a transfer fraction is formed. At least part of the transfer fraction is transferred to the steam cracking or the crude gas compression.

### CROSS-REFERENCE TO RELATED APPLICATIONS

This application is the national phase of, and claims priority to, International Application No. PCT/EP2021/080124, filed 29 Oct. 2021, which claims priority to EP Application No. 20204783.3, filed 29 Oct. 2020.

### FIELD OF THE INVENTION

The invention relates to a process and a plant for the production of olefins.

### BACKGROUND

The production of propylene from propane by dehydrogenation (propane dehydrogenation, PDH) is well known and is a commercially available and established process in the chemical industry. For an overview, reference is made, e.g., to the article “Propene” in Ullmann's Encyclopedia of Industrial Chemistry, 2013, DOI: 10.1002/1435007.a22_211.pub3, especially chapter 3.3.1, “Propane Dehydrogenation”.

EP 3 428 143 A1 relates to a process for the production of propylene which comprises carrying out a propane dehydrogenation process to obtain a first mixture of components, carrying out a steam cracking process to obtain a second mixture of components, forming a first separation product containing at least predominantly propylene using one or more first separation steps, forming a second separation product comprising at least predominantly propane using said first separation step or steps, forming a third separation product comprising at least predominantly ethylene using said second separation step or steps, and forming a fourth separation product comprising at least predominantly ethane using said second separation step or steps. It is provided that at least a portion of the first component mixture is subjected to one or more first pre-separation steps comprising a pressure increase and an at least partial removal of hydrogen to obtain a third component mixture, that at least a portion of the second component mixture is subjected to one or more second pre-separation steps to obtain a fourth component mixture, comprising an increase of pressure, an at least partial removal of hydrogen and an at least partial removal of methane, and in that at least a part of the third component mixture is subjected together with at least a part of the fourth component mixture to the first separation step or steps. A corresponding plant and method for converting a steam cracking plant is also an object of the invention.

Propane dehydrogenation is typically characterised by a very high selectivity to the main product propylene. Essential further components in the product gas are unreacted propane and hydrogen. The carbon-containing secondary components are predominantly ethane and/or ethylene and methane and hydrocarbons with four or more carbon atoms.

It would be desirable to be able to use at least a part of the mentioned secondary components in an economically advantageous way.

### SUMMARY

According to an embodiment of the invention, a process for the production of olefins includes subjecting a first feed stream comprising propane and hydrogen to propane dehydrogenation to obtain a first product stream. The first product includes at least propylene, propane, ethane and/or ethylene, methane and hydrogen. The process continues by subjecting at least part of the first product stream to a first separation sequence to form a transfer fraction that includes at least ethane and/or ethylene. The first separation sequence includes comprises a first separation step and a second separate step. Moving on, a second feed stream is subjected to steam cracking to obtain a second product stream, and at least part of the second product stream is subjected to crude gas compression and thereafter to a second separation sequence. The first separation step includes receiving at least part of the first product stream; and forming a gas fraction enriched in hydrogen and methane and a liquid fraction depleted in hydrogen and methane. The second separation step includes receiving at least part of the liquid fraction and forming the transfer fraction. At least a part of the transfer fraction is transferred to the steam cracking or the crude gas compression.

### BRIEF DESCRIPTION OF THE DRAWINGS

The figure illustrates a process for the production of olefins according to an embodiment of the invention.

### Written Description

In the process for the production of olefins proposed according to the invention, a first feed stream comprising propane and hydrogen is subjected to propane dehydrogenation to obtain a first product stream comprising at least propylene, propane, ethane and/or ethylene, methane and hydrogen, and at least part of the first product stream is subjected to a first separation sequence. According to the invention, a second feed stream is subjected to steam cracking to obtain a second product stream and at least a portion of the second product stream is subjected to crude gas compression and thereafter to a second separation sequence. In the first separation sequence, according to the invention, a transfer fraction containing at least ethane and/or ethylene is formed, and at least a part of the transfer fraction is transferred to the steam cracking or the crude gas compression.

Depending on the process variant, a gas mixture can be formed in the propane dehydrogenation and possibly downstream steps as explained below which contains significantly more ethane than ethylene or vice versa, which is expressed here by the general formulation that the gas mixture “contains ethane and/or ethylene”. The invention is suitable for both variants. In the case of a gas mixture containing more ethane than ethylene, a transfer fraction with corresponding contents can be fed in particular into the steam cracking, in the case of a gas mixture containing more ethylene than ethane, in particular into the downstream crude gas compression.

In this way, the invention creates a process that enables the saving of fresh feedstock for steam cracking without additional equipment. The invention can be used in particular when propane dehydrogenation and steam cracking are planned in close proximity to each other or are to be realised by means of corresponding plant components.

In some propane dehydrogenation processes (for example, the so-called Oleflex process from UOP), a gas fraction and a liquid fraction are generated first. The gas fraction contains most of the hydrogen from the propane dehydrogenation product stream (referred to here as the “first” product stream). It also contains a large part of the methane from the propane dehydrogenation product stream as well as small amounts of hydrocarbons with two and three carbon atoms. The liquid fraction again contains the majority of the hydrocarbons with two and three carbon atoms and only small amounts of the hydrogen and methane. Furthermore, a heavy fraction with hydrocarbons with four and more carbon atoms can be formed.

The invention provides just this, so that the first separation sequence according to the invention comprises a first separation step, to which at least part of the first product stream is fed, and in which a gas fraction enriched in hydrogen and methane and a liquid fraction depleted in hydrogen and methane are formed, and in which the first separation sequence comprises a second separation step, to which at least part of the liquid fraction is fed, and in which the transfer fraction is formed. The terms “enriched” and “depleted” refer to a respective content in the first product stream, with a content of more than 1.5, 2, 5 or 10 times being referred to as “enrichment” and a content of less than 0.5, 0.25 or 0.1 times being referred to as “depletion”.

In a subsequent step, the liquid fraction is separated between hydrocarbons with two and three carbon atoms, typically at a pressure of between 20 and 35 bar (abs.). Accordingly, a gaseous product with hydrocarbons with two hydrocarbons and possibly lighter components is then present, which contains only small amounts of methane, hydrogen and possibly traces of carbon monoxide, carbon dioxide and acetylene. Depending on the design of the propane dehydrogenation, the proportion of hydrocarbons with two carbon atoms, as mentioned, consists either predominantly of ethane or of ethylene. The term “rich” or “predominantly” is intended herein to denote a content of more than 50%, 60%, 70%, 80% or 90%, and the term “small amounts” is intended to denote a content of less than 10%, 5% or 1%, the percentages herein each being intended to denote mole fractions, unless otherwise specified.

In the process according to the invention, in a corresponding embodiment, the second separation step is thus carried out at a pressure level of 20 to 35 bar (abs.). The transfer fraction may be formed in the second separation step such that it contains 0.01 to 1.5% hydrogen, 0 to 0.1% carbon monoxide, 0 to 0.015% carbon dioxide, 5 to 25% methane, 0 to 0.1% acetylene, 60 to 90% ethane and/or ethylene and 0.05 to 0.15% hydrocarbons having three carbon atoms. In one specific example, 1% hydrogen, 0.07% carbon monoxide, 0.01% carbon dioxide, 16% methane, 0.02% acetylene, 7% ethylene, 76% ethane and 0.1% hydrocarbons with three carbon atoms are present. This specific example therefore concerns the case where a gas mixture with more ethane than ethylene is formed in the propane dehydrogenation and therefore the transfer fraction also has a higher ethane than ethylene content.

As mentioned, the process can be realised within the scope of the invention in the form of two process alternatives, i.e. feeding the transfer stream into the steam cracking (“variant 1”) and into the raw gas compression downstream of the steam cracking and upstream of the separation sequence (“variant 2”). The choice of the respective process alternatives depends, as mentioned, in particular on the ethane content in the transfer fraction.

Due to the typically high pressure at which the transfer stream, which advantageously contains more ethane than ethylene, is formed, it can be fed directly to a cracking furnace in variant 1 without further compression (possibly via preheating), since the component ethane intended for cracking is already present in the transfer stream in a high concentration. Furthermore, it can be assumed that none of the other components in the expected concentration will have a significant negative impact on the cracking process. It can also be assumed that the ethylene contained will survive the cracking process to a considerable extent and can also be recovered as a product. This variant hardly results in an additional load on the separation part and other feedstock for the cracking process can be saved in a corresponding amount. This variant is therefore particularly preferred if the transfer stream contains more ethane than ethylene, such as in the specific example mentioned above.

With variant 2, it can be ruled out that the cracking process is negatively influenced by other components. Variant 2 is particularly suitable for the cases mentioned where the transfer stream contains more ethylene than ethane. The stream is fed to the raw gas compression and in particular before the caustic wash, which by definition should be part of the second separation sequence here. This ensures that any small traces of, for example, carbon dioxide are still removed. Together with the cracked gas, the transfer stream is compressed here in the compression after the caustic wash to the final raw gas compressor pressure. Acetylenes from the transfer stream (if contained in traces) are hydrogenated in the following hydrogenation. Methane and possibly lighter components are removed in a corresponding separation step in the first separation sequence, hydrocarbons with three or more carbon atoms in a downstream separation step. Ethylene can be recovered in a so-called C2 splitter. It does not matter in which order the hydrogenation and the mentioned separation steps take place. Separated ethane from the transfer stream and from the second product stream can be returned to the cracking process, so that a corresponding amount of input is also saved in this way.

In technologies other than those explained above, the first product stream is not necessarily pre-separated into a gas fraction and a liquid fraction. Instead, the entire first product stream is separated directly between two and three carbon atoms. In this case, it would not make sense to run a correspondingly obtained lighter fraction directly to steam cracking as described before, since the overhead product can also be very rich in hydrogen and methane. In the case that this overhead product is first used for hydrogen recovery in a pressure swing adsorption, the tail gas of the pressure swing adsorption enriched with ethane and/or ethylene can advantageously be fed to the raw gas compression downstream of the steam cracking. Since the tail gas of the pressure swing adsorption has a low pressure of typically 1.2 to 8 bar (abs.), it is less advantageous to feed it directly into the steam cracking unit.

In the embodiments explained, a hydrogen- and methane-rich fraction is thus formed in the first separation sequence, at least part of which is used in unchanged composition as the transfer fraction, or from at least part of which the transfer fraction is formed, in particular using pressure swing adsorption.

With suitable pressure, in all embodiments the transfer fraction is fed to the steam cracking without further compression, so that additional compression equipment can be saved.

The invention also extends to a use of a plant for the production of olefins which is adapted to subject a first feed stream containing propane and hydrogen to propane dehydrogenation to obtain a first product stream containing at least propylene, propane, ethane and/or ethylene, methane and hydrogen, and which is adapted to subject at least part of the first product stream to a first separation sequence, wherein the first separation sequence comprises a first separation step to which at least part of the first product stream is fed and in which a gas fraction enriched in hydrogen and methane and a liquid fraction depleted in hydrogen and methane are formed, and wherein the first separation sequence comprises a second separation step to which at least part of the liquid fraction is fed and in which the transfer fraction is formed. Further, the plant comprises means adapted to subject a second feed stream to steam cracking to obtain a second product stream, to subject at least part of the second product stream to crude gas compression and thereafter to a second separation sequence, to form in the first separation sequence a transfer fraction comprising at least ethane and/or ethylene, and to transfer at least part of the transfer fraction to the steam cracking or the crude gas compression. According to the invention, the use comprises use in a process according to one embodiment of the invention.

With regard to the use proposed according to the invention, reference is therefore expressly made to the above explanations relating to the process according to the invention, since these concern a corresponding use in the same way. The same applies to an embodiment thereof.

The invention is further explained below with reference to the figure, which illustrates one embodiment of the invention.

Where reference is made below to process steps, the corresponding explanations apply equally to plant components with which these process steps are carried out, and vice versa.

The figure illustrates a process 100 for the production of olefins according to one embodiment of the invention. In the process 100, a first feed stream A containing propane and hydrogen is subjected to a propane dehydrogenation 10 to obtain a first product stream B containing at least propylene, propane, ethane, methane and hydrogen. At least part of the first product stream B is subjected to a first separation sequence 11, 12 comprising a first and a second separation step.

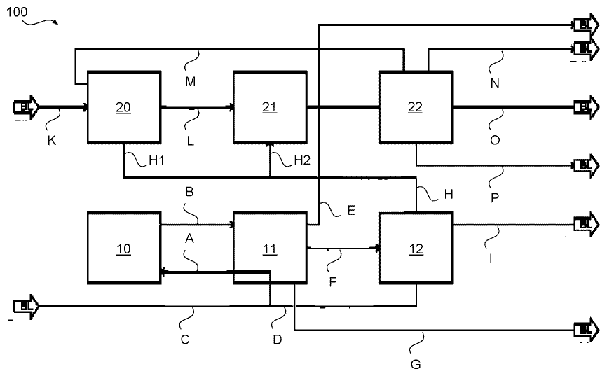

In the first separation step 11, a gas fraction enriched in hydrogen and methane and a liquid fraction depleted in hydrogen and methane are formed. Part of the hydrogen of the gas fraction is combined with a propane feed C and a propane recycle D to form the first feed stream A. Another part is sent to the plant boundary (referred to with BL throughout FIG. 1) in the form of a substance stream E. The liquid fraction is fed in the form of a substance stream F to the second separation step 12. In the first separation step 11, a fraction with hydrocarbons with four or more carbon atoms is also formed and fed to the plant boundary BL in the form of a material flow G. The material flow G is then fed to the plant boundary BL.

The liquid fraction contains in particular ethane and/or ethylene, propane and propylene as well as lighter components not transferred into the gas fraction separated in the first separation step 11. In the second separation step, a transfer fraction is thereby formed, which contains ethane and/or ethylene and possibly the lighter components, and which is carried out in the form of a substance stream H from the second separation step 12. Furthermore, a propylene product fraction is exported from the second separation step 12 in the form of a substance stream I to the plant boundary BL, as is a propane fraction which is provided in the form of the recycling stream D mentioned earlier.

A second feed stream K is subjected to steam cracking 20 to obtain a second product stream L. At least part of the second product stream L is subjected to a raw gas compression 21 and then to a second separation sequence 22, the latter being illustrated here in the form of a single functional block but may comprise different separation and processing steps. The transfer fraction H is transferred to the steam cracking 20 or the raw gas compression 21, as illustrated here in the form of alternative streams H1 and H2.

In the second separation sequence, in the exemplary embodiment shown here, a paraffin fraction is formed which is recycled to the steam cracking unit 20 in the form of a recycle stream M, a tail gas fraction is formed which is fed to the unit boundary BL in the form of a substance stream N, an ethylene product fraction is formed which is fed to the unit boundary BL in the form of a substance stream O, and a fraction containing hydrocarbons with three or more carbon atoms is formed which is fed to the unit boundary BL in the form of a substance stream P.

1. A process for the production of olefins comprising:
subjecting a first feed stream comprising propane and hydrogen to propane dehydrogenation to obtain a first product stream, the first product comprising at least propylene, propane, ethane and/or ethylene, methane and hydrogen;
subjecting at least part of the first product stream to a first separation sequence to form a transfer fraction comprising at least ethane and/or ethylene, wherein the first separation sequence comprises a first separation step and a second separate step;
subjecting a second feed stream to steam cracking to obtain a second product stream;
subjecting at least part of the second product stream to crude gas compression and thereafter to a second separation sequence;
wherein:
the first separation step comprises receiving at least part of the first product streams and forming a gas fraction enriched in hydrogen and methane and a liquid fraction depleted in hydrogen and methane; and
the second separation step comprises receiving at least part of the liquid fraction; and forming the transfer fraction;
and
at least a part of the transfer fraction is transferred to the steam cracking or the crude gas compression.

2. The process according to claim 1, in which the second separation step is carried out at a pressure level of 20 to 35 bar (abs.).

3. The process according to claim 1, wherein the transfer fraction contains 0.01 to 1.5% hydrogen, 0 to 0.1% carbon monoxide, 0 to 0.015% carbon dioxide, 5 to 25% methane, 0 to 0.1% acetylene, 60 to 90% ethane and/or ethylene and 0.05 to 0.15% hydrocarbons having three carbon atoms.

4. The process according to claim 1, further comprising forming, in the first separation sequence, a fraction rich in hydrogen and methane and containing ethane and/or ethylene, at least part of which is used in unchanged composition as the transfer fraction, or from at least part of which the transfer fraction is formed.

5. The process according to claim 4, wherein the transfer fraction is formed using pressure swing adsorption from at least a portion of the fraction containing hydrogen and methane and ethane and/or ethylene.

6. The process according to claim 1, in which the transfer fraction is fed to the steam cracking without further compression.

7. (canceled)

8. The process according to claim 2, wherein the transfer fraction contains 0.01 to 1.5% hydrogen, 0 to 0.1% carbon monoxide, 0 to 0.015% carbon dioxide, 5 to 25% methane, 0 to acetylene, 60 to 90% ethane and/or ethylene and 0.05 to 0.15% hydrocarbons having three carbon atoms.

9. The process according to claim 8, in which the transfer fraction is fed to the steam cracking without further compression.

10. The process according to claim 2, in which the transfer fraction is fed to the steam cracking without further compression.

11. The process according to claim 3, in which the transfer fraction is fed to the steam cracking without further compression.

12. The process according to claim 4, in which the transfer fraction is fed to the steam cracking without further compression.

13. The process according to claim 5, in which the transfer fraction is fed to the steam cracking without further compression.
